(19) United States
(12) Patent Application Publication
(10) Pub. No.: US 2020/0131501 A1
(43) Pub. Date: Apr. 30, 2020

(54) METHOD AND COMPOSITION FOR PRODUCING TARGET NUCLEIC ACID MOLECULE

(71) Applicants: CELEMICS, INC., Seoul (KR); SEOUL NATIONAL UNIVERSITY R&DB FOUNDATION, Seoul (KR)

(72) Inventors: Sung Hoon KWON, Seoul (KR); Jung Min KIM, Seoul (KR); Jin Sung NOH, Seoul (KR); Hui Ran YEOM, Cheorwon-gun (KR); Tae Hoon RYU, Gimpo-si (KR)

(21) Appl. No.: 16/486,668

(22) PCT Filed: Feb. 20, 2018
(86) PCT No.: PCT/KR2018/002049

§ 371 Date: Aug. 16, 2019

(30) Foreign Application Priority Data
Feb. 20, 2017 (KR) 10-2017-0022167

(51) Int. Cl. C12N 15/10; C12N 9/22; C12N 9/12

(52) U.S. Cl. CPC C12N 15/10; C12N 9/22; C12Y 207/07007; C12N 2800/80; C12N 9/1252

(57) ABSTRACT

The present invention provides a method for producing a target nucleic acid molecule, including: (a) providing a double-stranded nucleic acid molecule including a target sequence region, a first flanking sequence region linked to the 5′ end of the target sequence region and containing one or more deaminated bases, and a second flanking sequence region linked to the 3′ end of the target sequence region; and (b) incubating the nucleic acid molecule and an endonuclease specific for the deaminated bases to remove the first flanking sequence region ranging from the deaminated base closest to the 5′ end of the target sequence region to the 5′ end of the nucleic acid molecule. The present invention also provides a composition for producing a target nucleic acid molecule including the double-stranded nucleic acid molecule and a deaminated base-specific endonuclease.

### TECHNICAL FIELD

The present invention relates to a method and composition for producing a target nucleic acid molecule.

### BACKGROUND ART

The concentration of products synthesized by current microarray-based gene synthesis techniques is at the femtomolar level. Thus, it is necessary to increase the concentration of the synthesized products to a higher level by PCR. In an attempt to meet this demand, generally, a primer-binding region is synthesized during gene synthesis and a restriction enzyme recognition sequence is introduced into the primer-binding region. The restriction enzyme recognition sequence is used for removal of the primer binding region in a subsequent process.

A restriction enzyme recognizes a specific nucleotide sequence and cleaves DNA in or around the sequence. The enzyme generally recognizes 4 to 8 bases in the sequence. The presence of a restriction enzyme recognition site in a target nucleic acid may be an obstacle to the isolation of the intact target nucleic acid. When it is desired to obtain a target nucleic acid in an intact form, the choice of a suitable restriction enzyme according to the synthetic sequence is troublesome.

When reaction products with a restriction enzyme need to be specially treated, time and cost problems may arise. Gene synthesis products are assembled into a longer nucleic acid molecule by gene assembly. Reaction products with a restriction enzyme may have sticky ends. In this case, an additional process is required to convert the sticky ends to blunt ends.

Under these circumstances, the present inventors have succeeded in designing a method for producing a target nucleic acid molecule by cleaving a nucleic acid in a sequence-independent manner.

### DETAILED DESCRIPTION OF THE INVENTION

### Problems to be Solved by the Invention

One aspect provides a method for producing a target nucleic acid molecule from a double-stranded nucleic acid molecule including a target sequence region, a first flanking sequence region linked to the 5′ end of the target sequence region and containing one or more deaminated bases, and a second flanking sequence region linked to the 3′ end of the target sequence region.

A further aspect provides a composition for producing a target nucleic acid molecule including the double-stranded nucleic acid molecule and a deaminated base-specific endonuclease.

### Means for Solving the Problems

One aspect provides a method for producing a target nucleic acid molecule, including: (a) providing a double-stranded nucleic acid molecule including a target sequence region, a first flanking sequence region linked to the 5′ end of the target sequence region and containing one or more deaminated bases, and a second flanking sequence region linked to the 3′ end of the target sequence region; and (b) incubating the nucleic acid molecule and an endonuclease specific for the deaminated bases to remove the first flanking sequence region ranging from the deaminated base closest to the 5′ end of the target sequence region to the 5′ end of the nucleic acid molecule.

The first flanking sequence region may have at least 2, at least 3 or at least 4 deaminated bases. In the first flanking sequence region, one or more nucleotides may be arranged between the adjacent deaminated bases. The deaminated bases may be inosine or uracil bases.

The deaminated bases may be inosine bases. In this case, 3 to 8 nucleotides may be arranged between the adjacent inosine bases. For example, when 3 inosine bases are present in the first flanking sequence region, the adjacent inosine bases may be separated by 5 and 8 nucleotides. When 4 inosine bases are present in the first flanking sequence region, the adjacent inosine bases may be separated by 4, 3, and 5 nucleotides. Alternatively, the deaminated bases may be uracil bases. In this case, one or more nucleotides may be arranged between the adjacent uracil bases.

At least one of the deaminated bases may be located at the first, second or third nucleotide from the 3′ end of the first flanking sequence region. For example, at least one of the deaminated bases may be present in the nucleotide at the 3′ end of the first flanking sequence region, the second nucleotide from the 3′ end of the first flanking sequence region or the third nucleotide from the 3′ end of the first flanking sequence region.

When the deaminated bases are inosine bases, the deaminated base-specific endonuclease may be endonuclease V, often called deoxyinosine 3′-endonuclease. Endonuclease V recognizes hypoxanthine, the base of deoxyinosine, on single- or double-stranded DNA and hydrolyzes mainly the second or third phosphodiester bond at the 3′ end of the recognized base to create “nicks”. The inosine-specific endonuclease may be endonuclease V derived from Thermotoga maritima or E. coli.

When the deaminated bases are uracil bases, the deaminated base-specific endonuclease may be a uracil-specific excision reagent (USER). USER is an enzyme that generates a single nucleotide gap at the location of a uracil residue. USER enzyme is a mixture of uracil DNA glycosylase (UDG) and DNA glycosylase-lyase endonuclease VIII. UDG catalyzes the excision of a uracil base, forming an abasic site while leaving the phosphodiester backbone intact. The lyase activity of endonuclease VIII breaks the phosphodiester backbone at the 3′ and 5′ sides of the abasic site so that base-free deoxyribose is released.

The double-stranded nucleic acid molecule may be a product obtained by amplifying a template nucleic acid molecule including the target sequence region, a third flanking sequence region linked to the 5′ end of the target sequence region, and a fourth flanking sequence region linked to the 3′ end of the target sequence region with a primer set containing one or more deaminated bases and annealing to the fourth flanking sequence region.

The template nucleic acid molecule may be one isolated from an organism, one isolated from a library of nucleic acids, one obtained by modifying or combining isolated nucleic acid fragments by genetic engineering, one obtained by chemical synthesis, or a combination thereof. The template nucleic acid molecule may be single- or double-stranded.

Alternatively, the template nucleic acid molecule may be prepared by microarray-based synthesis. Microarray-based synthesis refers to a technique for simultaneous parallel synthesis of identical, similar or different types of biochemical molecules on synthetic spots immobilized at intervals in the centimeter or micrometer range on a solid substrate.

The primer set for amplifying the template nucleic acid molecule may be annealed to the fourth flanking sequence region of the template nucleic acid molecule and may have at least 2, at least 3 or at least 4 deaminated bases. The deaminated bases may be inosine or uracil bases.

The deaminated bases may be inosine bases. In this case, 3 to 8 nucleotides may be arranged between the adjacent inosine bases. For example, when 3 inosine bases are present in the first flanking sequence region, the adjacent inosine bases may be separated by 5 and 8 nucleotides. When 4 inosine bases are present in the first flanking sequence region, the adjacent inosine bases may be separated by 4, 3, and 5 nucleotides. Alternatively, the deaminated bases may be uracil bases. In this case, one or more nucleotides may be arranged between the adjacent uracil bases.

The primer set may be a pair of an oligonucleotide having the nucleotide sequence set forth in SEQ ID NO: 1 and an oligonucleotide having the nucleotide sequence set forth in SEQ ID NO: 2, a pair of an oligonucleotide having the nucleotide sequence set forth in SEQ ID NO: 3 and an oligonucleotide having the nucleotide sequence set forth in SEQ ID NO: 4, a pair of an oligonucleotide having the nucleotide sequence set forth in SEQ ID NO: 5 and an oligonucleotide having the nucleotide sequence set forth in SEQ ID NO: 6, a pair of an oligonucleotide having the nucleotide sequence set forth in SEQ ID NO: 7 and an oligonucleotide having the nucleotide sequence set forth in SEQ ID NO: 8 or a pair of an oligonucleotide having the nucleotide sequence set forth in SEQ ID NO: 15 and an oligonucleotide having the nucleotide sequence set forth in SEQ ID NO: 16.

The method may further include (c) incubating the nucleic acid molecule free of the first flanking sequence region and a 3′→5′ exonuclease to remove the single-stranded second flanking sequence region. The exonuclease may be T4 DNA polymerase.

Step (b) and (c) may be carried out by a one-shot process. According to the one-shot process, the reactants including the double-stranded nucleic acid molecule, the deaminated base-specific endonuclease, and the exonuclease are incubated at a higher temperature (step (b)), followed by incubation at a lower temperature (step (c)).

For example, the reactants including the double-stranded nucleic acid molecule, the deaminated base-specific endonuclease, and the exonuclease may be incubated at 36° C. to 65° C., 38° C. to 60° C., 40° C. to 58° C., 40° C. to 55° C. or 40° C. to 50° C. for 20 minutes to 40 minutes or 25 minutes to 35 minutes, for example, 30 minutes, followed by incubation at 20° C. to 30° C., 22° C. to 28° C. or 23.5° C. to 26.5° C. for 15 minutes to 25 minutes or 18 minutes to 23 minutes, for example 20 minutes.

A further aspect provides a composition for producing a target nucleic acid molecule, including: a double-stranded nucleic acid molecule including a target sequence region, a first flanking sequence region linked to the 5′ end of the target sequence region and containing one or more deaminated bases, and a second flanking sequence region linked to the 3′ end of the target sequence region; and an endonuclease specific for the deaminated bases.

The first flanking sequence region may have at least 2, at least 3 or at least 4 deaminated bases. In the first flanking sequence region, one or more nucleotides may be arranged between the adjacent deaminated bases. The deaminated bases may be inosine or uracil bases. The deaminated bases may be inosine bases. In this case, 3 to 8 nucleotides may be arranged between the adjacent inosine bases. Alternatively, the deaminated bases may be uracil bases. In this case, one or more nucleotides may be arranged between the adjacent uracil bases. At least one of the deaminated bases may be located at the first, second or third nucleotide from the 3′ end of the first flanking sequence region.

When the deaminated bases are inosine bases, the deaminated base-specific endonuclease may be endonuclease V. The endonuclease V is the same as that described above. When the deaminated bases are uracil bases, the deaminated base-specific endonuclease may be a uracil-specific excision reagent (USER). The uracil-specific excision reagent is the same as that described above.

The double-stranded nucleic acid molecule may be a product obtained by amplifying a template nucleic acid molecule including the target sequence region, a third flanking sequence region linked to the 5′ end of the target sequence region, and a fourth flanking sequence region linked to the 3′ end of the target sequence region with a primer set containing one or more deaminated bases and annealing to the fourth flanking sequence region. The template nucleic acid molecule is the same as that described above. Alternatively, the template nucleic acid molecule may be prepared by microarray-based synthesis. The primer set for amplifying the template nucleic acid molecule is the same as that described above.

The composition may further include a 3′→5′ exonuclease. The exonuclease may be T4 DNA polymerase.

### Effects of the Invention

The method and composition for producing a target nucleic acid molecule according to aspects can be widely utilized in the fields of synthetic biology and molecular biology.

### BRIEF DESCRIPTION OF THE DRAWINGS

FIG. 1a is a schematic diagram showing a method for producing a target nucleic acid molecule according to one aspect.

FIG. 1b shows a process for preparing a double-stranded nucleic acid molecule in a method for producing a target nucleic acid molecule according to one aspect.

FIG. 2a shows the results of electrophoresis in individual steps using inosine-containing primers and restriction enzymes.

FIG. 2b shows the results of electrophoresis in individual steps using uracil-containing primers and restriction enzymes.

FIG. 3 shows the activities of two enzymes at various temperatures.

FIG. 4 shows the results of experiments to determine whether or not a purification process may be omitted and a buffer mixture may be used.

FIG. 5a is a schematic diagram showing a one-shot reaction according to one aspect.

FIG. 5b shows the results obtained after a one-shot reaction at various temperatures.

### MODE FOR CARRYING OUT THE INVENTION

The present invention will be explained in more detail with reference to the following examples. However, these examples are provided to assist in understanding the present invention and are in no way intended to limit the scope of the invention.

### EXAMPLE 1

### Cleavage with Inosine-Containing Primers

1.1. Preparation of DNA Fragments and Primer Sets and PCR

257 140-bp single-stranded DNA fragments were prepared from the genomic DNA of Mycoplasma genitalium using a semiconductor-based electrochemical acid production array (CustomArray). Each fragment had the common sequences (SEQ ID NOS: 9 and 10) for primer annealing that flank a target sequence. The 257 fragments were categorized into 20 sets of cassettes according to an 80-bp overlapping region located in the 100 bp target sequence.

Primer sets that can be annealed to the common sequences were prepared. The sequences of the primer sets were identical to the common sequences except that one or more guanine bases in the common sequences were replaced by inosine bases. The primer sets were named CP primer sets. All primers were customized by Integrated DNA Technology (Coralvile, Iowa, USA). The CP primer sets are shown in Table 1.

TABLE 1

| Primer name | Primer sequence |
|---|---|
| CP 1 Forward | 5′-GTG CCT TGG CAG TCT CAI T-3′ |
| | (19 bp) |
| CP 1 Reverse | 5′-CGT GGA TGA GGA GCC GCA GTI |
| | T-3′ (22 bp) |
| CP 2 Forward | 5′-GTI CCT TG I CAG TCT CAI T-3′ |
| | (19 bp) |
| CP 2 Reverse | 5′-CGT GI A TGA GGA ICC GCA GTI |
| | T-3′ (22 bp) |
| CP 3 Forward | 5′-GTI CCT TGI CAG TCT CA |
| | 3deoxy-3′ (18 bp) |
| CP 3 Reverse | 5′-CGT GI A TGA GGA ICC GCA GT |
| | 3deoxy-3′ (21 bp) |
| CP 4 Forward | 5′-GTI CCT TIG CA I TCT CAI T-3′ |
| | (19 bp) |
| CP 4 Reverse | 5′-T GIA TGA GIA GCC ICA GTI T-3′ |
| | (20 bp) |

As shown in Table 1, each of the CP 1 primers (SEQ ID NOS: 1 and 2) had one inosine base in front of thymine at the 3′ end. Each of the CP 2 primers (SEQ ID NOS: 3 and 4) and the CP 3 primers (SEQ ID NOS: 5 and 6) had three inosine bases. The adjacent inosine bases in each of the CP 2 and CP 3 primers were separated by 5 and 8 nucleotides. Each of the CP 3 primers has deoxyinosine at the 3′ end, unlike the CP 2 primers. Each of the CP 4 primers (SEQ ID NOS: 7 and 8) has four inosine bases. The adjacent inosine bases were separated by 4, 3, and 5 nucleotides.

PCR of the DNA fragments was performed using Taq DNA polymerase (Thermo Scientific) with the CP primer sets. Specifically, a solution (50 μl) containing 700 ng of M. genitalium genomic DNA and 1 pM of each of the CP primer sets was allowed to react at 95° C. for 2 min. After 10-15 cycles consisting of 95° C./30 sec, annealing temperature/20 sec, and 72° C./30 sec, the reaction was continued at 72° C. for 2 min. The annealing temperature varied depending on the primer type. The reaction products were purified using a QIAGEN MinElute PCR purification kit (QIAGEN, Valencia, Calif., USA) and eluted to a final volume of 15 μl.

1.2. Reactions with Endonuclease and Exonuclease and Sequencing

700 ng of each of the DNA-containing purified PCR products and endonuclease V (Thermo Fisher Scientific, St. Leon-Rot Germany, 5 U/μl) derived from Thermotoga maritima (Tma) were incubated at 65° C. for 30 min, purified, and eluted to a final volume of 15 μl.

Thereafter, the eluate was allowed to react with T4 DNA polymerase (Thermo Scientific, 5 U/μl) having a 3′→5′ exonuclease activity at 11° C. for 20 min or at room temperature for 5 min. High-resolution electrophoresis was performed in 2.5% agarose gels at 120 V for 60-90 min to determine the sizes and amounts of the DNA fragments.

For sequencing, the phosphate residues at the 5′ and 3′ ends were removed by treatment with alkaline phosphatase (Calf Intestinal; New England Biolabs). TOPO cloning of 1 μl of 20 ng/μl DNA from which the phosphate residues at both ends had been removed was performed with an All in One PCR cloning kit (Biofact) according to the manufacturer's instructions, followed by Sanger sequencing (Macrogen Inc.). To obtain sequence information on a large number of colonies at low cost, all colonies were collected in one tube, cells were cultured in liquid LB medium, and then plasmids were purified with a Geneall Exprep plasmid mini kit. Primers were designed from the flanking sequences of the cloning sites of the plasmids such that a target sequence was present in the amplification products. The amplification products were requested for sequencing by Illumina Mi Seq. Sequencing results were obtained for tens of thousands of templates.

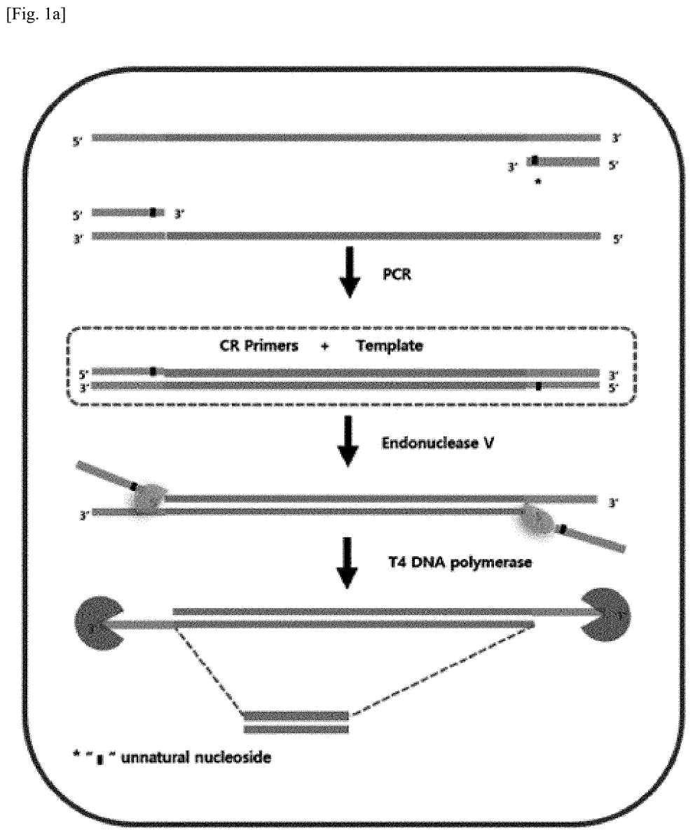

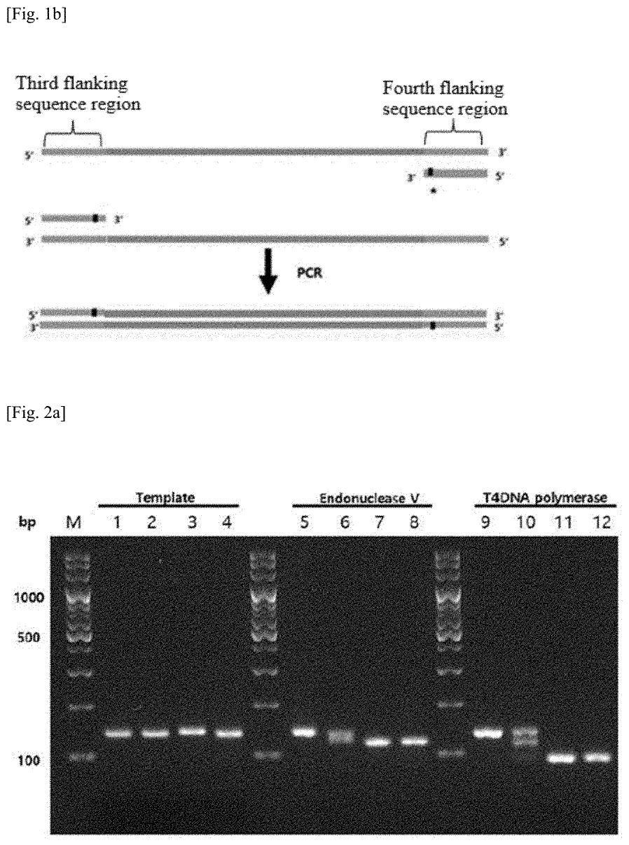

FIG. 1a is a schematic diagram showing a method for producing a target nucleic acid molecule according to one aspect.

FIG. 1b shows a process for preparing a double-stranded nucleic acid molecule in a method for producing a target nucleic acid molecule according to one aspect. As shown in FIG. 1b, a template nucleic acid molecule includes a third flanking sequence region linked to the 5′ end of a target sequence region and a fourth flanking sequence region linked to the 3′ end of the target sequence region. A primer set containing deaminated bases is annealed to the fourth flanking sequence region. This annealing enables the amplification of the template nucleic acid molecule.

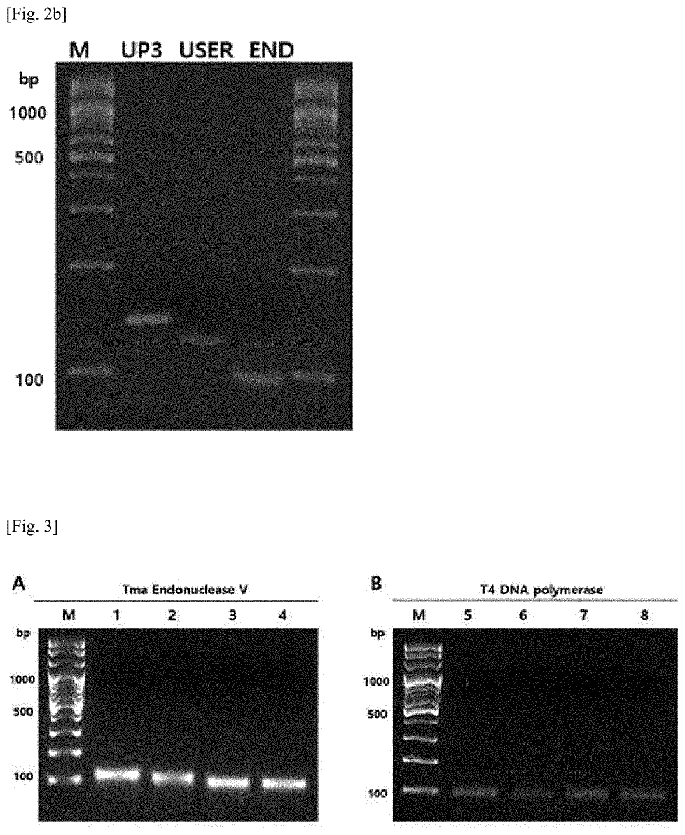

FIG. 2a shows the results of electrophoresis in individual steps using inosine-containing primers and restriction enzymes. Lanes 1-4 represent PCR products using the common primer set, the CP 1 primer set, the CP 2 primer set, and the CP 3 primer set, respectively. Lanes 5-8 represent products obtained by reactions of the PCR products in Lanes 1-4 with Tma Endo V, respectively. Lanes 9-12 represent products obtained by reactions of the products in Lanes 5-8 with T4 DNA polymerase, respectively.

As shown in FIG. 2a, for the CP 1 primer set (Lane 10), cleaved fragments and non-cleaved fragments coexisted. For the CP 2 and CP 3 primer sets (Lanes 11 and 12), 100-bp final products were generated. Sanger sequencing of the final products revealed that 73.7% (CP 2) and 93.8% (CP 3) were cleaved.

For the CP 4 primers, Sanger sequencing revealed 100% cleavage. The cleavage performance was again confirmed by Illumina Mi-Seq. The results are shown in Table 2. In Table 2, F and R represent the forward and reverse primers, respectively, and Cut and Uncut represent the numbers of cleaved and non-cleaved reads at the inosine bases of the primers, respectively. Sample 1 and Sample 2 are two parallel experimental groups treated under the same conditions. As a result of Illumina sequencing for Sample 1, F and R primer sites were accurately cleaved in a total of 95365 reads, the target sequence only remained in 94631 reads, the R primer was not cleaved in 732 reads, and the F primer was not cleaved in 2 reads. Illumina sequencing for Sample 2 revealed that the F and R primers were accurately cleaved and the target sequence only remained in 88649 reads, the R primer was not cleaved in 361 reads, and the F primer was not cleaved in 815 reads. As shown in Table 2, 98.97% of the templates were successfully cleaved. The remainder (1.03%) is estimated due to errors during the primer construction. These results concluded that the inventive method is effective also in large-scale experiments.

TABLE 2

| Sample | | R | | Sample | | R | |
|---|---|---|---|---|---|---|---|
| 1 | | Cut | Uncut | 2 | | Cut | Uncut |
| F | Cut | 94631 | 732 | F | Cut | 88649 | 361 |
| | Un- | 2 | 0 | | Un- | 815 | 1 |
| | cut | | | | cut | | |

### EXAMPLE 2

### Cleavage with Uracil-Containing Primers

2.1. Preparation of Primer Sets and PCR

PCR of DNA fragments derived from Mycoplasma genitalium described in Example 1 as templates was performed with the primer sets having the sequences shown in Table 3. Each of the primer sets included one or more uracil bases. The primer sets were named UP primer sets. The UP primer sets are shown in Table 3.

TABLE 3

| Primer name | Primer sequence |
|---|---|
| UP 1 Forward | 5′-GTG CCT TGG CAG TCT CAG U-3′ |
| | (19 bp) |
| UP 1 Reverse | 5′-CGT GGA TGA GGA GCC GCA GTG U-3′ |
| | (22 bp) |
| UP 2 Forward | 5′-GTG CCT TGG CAG TCU CAG T-3′ |
| | (19 bp) |
| UP 2 Reverse | 5′-CGT GGA TGA GGA GCC GCA GUG T-3′ |
| | (22 bp) |
| UP 3 Forward | 5′-GTG CCU TGG CAG UCT CAG-3′ |
| | (18 bp) |
| UP 3 Reverse | 5′-CGT GGA UGA GGA GCU GCA GTG-3′ |
| | (21 bp) |

As shown in Table 3, each of the UP 1 primers (SEQ ID NOS: 11 and 12) had one uracil base at the 3′ end and each of the UP 2 primers (SEQ ID NOS: 13 and 14) had one uracil base at the fifth or third position from the 3′ end. In each of the UP 3 primers (SEQ ID NOS: 15 and 16), 6 or 7 nucleotides were arranged between two uracil bases.

A solution (50 μl) containing 1 μl of 10 μM M. genitalium genomic DNA, 25 pmol of each of the UP primer sets, and 25 μl of KAPA HiFi HotStart Uracil+ReadyMix (2×) was allowed to react at 95° C. for 2 min. After 11 cycles consisting of 98° C./20 sec, 58° C./15 sec, and 72° C./30 sec, the reaction was continued at 72° C. for 2 min. The sizes of the PCR products were constant at 140 bp.

2.2. Reactions with Endonuclease and Exonuclease and Sequencing

A solution (100 μl) containing 50 μl of each of the PCR products obtained in 2.1, 10 μl of 10× CutSmart buffer, and 10 μl of USER enzyme (NEB) was incubated at 37° C. for 20 min, purified, and eluted to a final volume of 12 μl. A solution (20 μl) containing 10 μl of the eluate, 2 μl of 10× End Repair reaction buffer, and 1 μl of End Repair enzyme mix (NEB) was allowed to react at 20° C. for 30 min, purified, and eluted to a final volume of 12 μl. High-resolution electrophoresis was performed in 2.5% agarose gels at 120 V for 60-90 min to determine the sizes and amounts of the DNA fragments.

FIG. 2b shows the results of electrophoresis in individual steps using uracil-containing primers and restriction enzymes. In FIG. 2b, UP3 represents the PCR products (140 bp) obtained using the uracil-containing primer set UP 3, USER represents the products cleaved by USER enzyme, and END represents the cleavage products (100 bp) that were blunt-ended by End Repair enzyme. Sanger sequencing for a total of 83 cleavage products revealed that 6 (7.2%) of the products had a length of 99 bp and 77 (92.8%) of the products had a length of 100 bp, demonstrating that all nucleic acid fragments were cleaved by the uracil-containing primers and USER enzyme.

### EXAMPLE 3

### Extension of Enzymatic Reaction Conditions

3.1. Test for Temperature-Dependent Activity

The activities of Tma Endo V and T4 DNA polymerase were tested under various temperature conditions.

FIG. 3 shows the activities of the two enzymes at various temperatures.

Tma Endo V was incubated at various temperatures as well as at the recommended incubation temperature (65° C.). Lanes 1-4 show the results of electrophoresis after each of the products obtained by incubation at 25° C., 35° C., 50° C., and 65° C. was allowed to react with T4 DNA polymerase at 25° C. for 20 min. As shown in A of FIG. 3, Tma Endo V showed no substantial activity at temperatures of ≤35° C. (Lanes 1 and 2). The activity of Tma Endo V was observed at 50° C. and 65° C.

The recommended incubation conditions for the 3′→5′ exonuclease activity of T4 DNA polymerase are 11° C./20 min or room temperature/5 min. As shown in B of FIG. 3, however, the activity of T4 DNA polymerase was maintained under various temperature conditions, including 25° C., 35° C., 50° C., and 65° C., as well as at the recommended incubation temperature (Lanes 5-8).

3.2. Test to Determine Whether or Not Purification Process may be Omitted and Buffer Mixture may be Used

PCR products are purified by removing unnecessary components, including salts, nucleotides, enzymes, and primers. Cleaning up of DNA samples is also required for subsequent enzymatic treatment. However, purification incurs enormous costs and presents a difficulty in complete automation in large-scale experiments. Accordingly, the omission of purification contributes to time and cost savings, thus being advantageous for an operator.

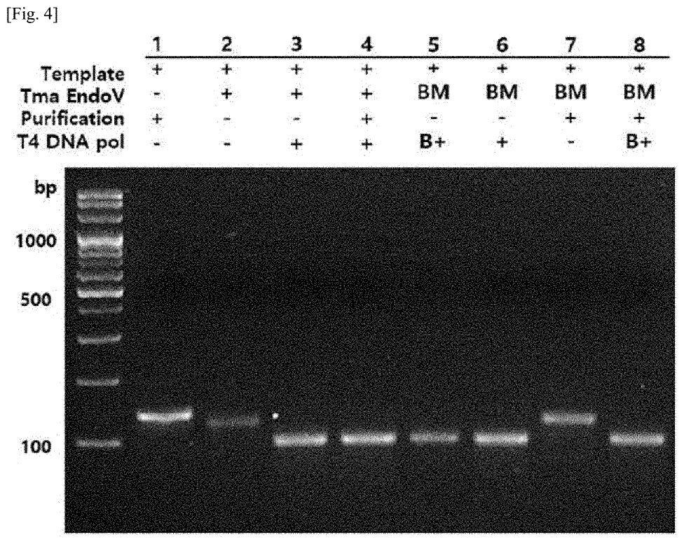

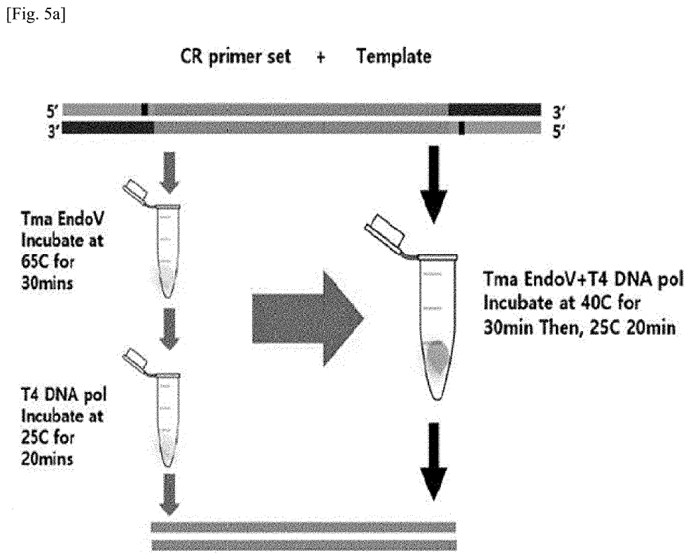

FIG. 4 shows the results of experiments to determine whether or not a purification process may be omitted and a buffer mixture may be used. 140-bp amplification products were obtained using the CP 3 primer set, treated with Tma Endo V (Lane 2), purified (Lane 4) or not purified (Lane 3), and treated with T4 DNA polymerase. For Lanes 5-8, after incubation in a buffer mixture (BM) of Tma Endo V buffer and T4 DNA polymerase buffer, the resulting reaction products were compared. B+ of FIG. 4 represents one-time addition of the T4 DNA polymerase buffer.

As shown in FIG. 4, comparison of Lanes 3 and 4 confirms that the omission of purification before the addition of T4 DNA polymerase did not affect cleavage. It was also confirmed that the use of the buffer mixture did not inhibit the activities of the two enzymes.

3.3. One-Shot Reaction

Considering that the omission of purification or the use of the buffer mixture has no influence on the activities of the enzymes, as confirmed in 3.2, a one-shot reaction with the two enzymes in a matrix was performed. Optimal temperature and time conditions for this reaction were investigated.

700 ng of a template matrix, 5 units of Tma Endo V, 1 μl of T4 DNA polymerase and dNTP, and a buffer mixture were mixed together to prepare 100 μl of a solution.

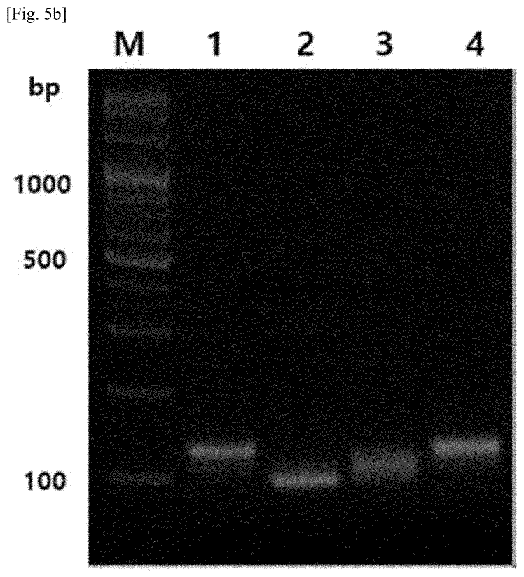

FIG. 5a is a schematic diagram showing a one-shot reaction according to one aspect.

FIG. 5b shows the results obtained after a one-shot reaction at various temperatures. Lanes 1-4 represent the results obtained after incubation at 50° C. for 30 min and subsequent incubation at 25° C. for 20 min (Lane 1), after incubation at 40° C. for 30 min and subsequent incubation at 25° C. for 20 min (Lane 2), after incubation at 40° C. for 30 min and subsequent incubation at 25° C. for 20 min (Lane 3), and after incubation at 45° C. for 30 min and subsequent incubation at 25° C. for 20 min (Lane 4). As shown in FIG. 5b, incubation at 40° C. for 30 min and subsequent incubation at 25° C. for 20 min were optimal one-shot reaction conditions.

1. A method for producing a target nucleic acid molecule, comprising: (a) providing a double-stranded nucleic acid molecule comprising a target sequence region, a first flanking sequence region linked to the 5′ end of the target sequence region and containing one or more deaminated bases, and a second flanking sequence region linked to the 3′ end of the target sequence region; and (b) incubating the nucleic acid molecule and an endonuclease specific for the deaminated bases to remove the first flanking sequence region ranging from the deaminated base closest to the 5′ end of the target sequence region to the 5′ end of the nucleic acid molecule.

2. The method according to claim 1, wherein the double-stranded nucleic acid molecule is a product obtained by amplifying a template nucleic acid molecule comprising the target sequence region, a third flanking sequence region linked to the 5′ end of the target sequence region, and a fourth flanking sequence region linked to the 3′ end of the target sequence region with a primer set containing one or more deaminated bases and annealing to the fourth flanking sequence region; and the template nucleic acid molecule is prepared by microarray-based synthesis.

3. The method according to claim 1, wherein one or more nucleotides are arranged between the adjacent deaminated bases.

4. The method according to claim 1, wherein the deaminated bases are inosine or uracil bases.

5. The method according to claim 1, wherein the deaminated bases are inosine bases and the inosine-specific endonuclease is endonuclease V.

6. The method according to claim 5, wherein the inosine-specific endonuclease is endonuclease V derived from Thermotoga maritima or E. coli.

7. The method according to claim 1, wherein the deaminated bases are uracil bases and the uracil-specific endonuclease is a uracil-specific excision reagent (USER).

8. The method according to claim 1, wherein 3 to 8 nucleotides are arranged between the adjacent inosine bases.

9. The method according to claim 1, further comprising (c) incubating the nucleic acid molecule free of the first flanking sequence region and a 3′→5′ exonuclease to remove the single-stranded second flanking sequence region.

10. The method according to claim 9, wherein the exonuclease is T4 DNA polymerase.

11. The method according to claim 9, wherein step (b) and (c) are carried out by a one-shot process and the reactants comprising the double-stranded nucleic acid molecule, the deaminated base-specific endonuclease, and the exonuclease are incubated at 36° C. to 65° C., followed by incubation at 20° C. to 30° C.

12. A composition for producing a target nucleic acid molecule, comprising: a double-stranded nucleic acid molecule comprising a target sequence region, a first flanking sequence region linked to the 5′ end of the target sequence region and containing one or more deaminated bases, and a second flanking sequence region linked to the 3′ end of the target sequence region; and an endonuclease specific for the deaminated bases.

13. The composition according to claim 12, wherein the double-stranded nucleic acid molecule is a product obtained by amplifying a template nucleic acid molecule comprising the target sequence region, a third flanking sequence region linked to the 5′ end of the target sequence region, and a fourth flanking sequence region linked to the 3′ end of the target sequence region with a primer set containing one or more deaminated bases and annealing to the fourth flanking sequence region; and the template nucleic acid molecule is prepared by microarray-based synthesis.

14. The composition according to claim 12, wherein one or more nucleotides are arranged between the adjacent deaminated bases in the double-stranded nucleic acid molecule.

15. The composition according to claim 12, wherein the deaminated bases are inosine or uracil bases.

16. The composition according to claim 12, wherein the deaminated bases are inosine bases and the deaminated base-specific endonuclease is endonuclease V.

17. The composition according to claim 16, wherein the deaminated base-specific endonuclease is endonuclease V derived from Thermotoga maritima or E. coli.

18. The composition according to claim 12, wherein the deaminated bases are uracil bases and the uracil-specific endonuclease is a uracil-specific excision reagent (USER).

19. The composition according to claim 12, wherein 3 to 8 nucleotides are arranged between the adjacent deaminated bases.

20. The composition according to claim 12, further comprising a 3′→5′ exonuclease.

21. The composition according to claim 20, wherein the exonuclease is T4 DNA polymerase.


SEQUENCE LISTING (16 sequences)

SEQ ID NO: 1 - DNA, 19 bp, Artificial Sequence
  Synthesized
  misc_feature (18)..(18): n is a, c, g, or t
gtgccttggcagtctcant

SEQ ID NO: 2 - DNA, 22 bp, Artificial Sequence
  Synthesized
  misc_feature (21)..(21): n is a, c, g, or t
cgtggatgaggagccgcagtnt

SEQ ID NO: 3 - DNA, 19 bp, Artificial Sequence
  Synthesized
  misc_feature (3)..(3): n is a, c, g, or t
  misc_feature (9)..(9): n is a, c, g, or t
  misc_feature (18)..(18): n is a, c, g, or t
gtnccttgncagtctcant

SEQ ID NO: 4 - DNA, 22 bp, Artificial Sequence
  Synthesized
  misc_feature (5)..(5): n is a, c, g, or t
  misc_feature (13)..(13): n is a, c, g, or t
  misc_feature (21)..(21): n is a, c, g, or t
cgtgnatgagganccgcagtnt

SEQ ID NO: 5 - DNA, 18 bp, Artificial Sequence
  Synthesized
  misc_feature (3)..(3): n is a, c, g, or t
  misc_feature (9)..(9): n is a, c, g, or t
  misc_feature (18)..(18): n is a, c, g, or t
gtnccttgncagtctcan

SEQ ID NO: 6 - DNA, 21 bp, Artificial Sequence
  Synthesized
  misc_feature (5)..(5): n is a, c, g, or t
  misc_feature (13)..(13): n is a, c, g, or t
  misc_feature (21)..(21): n is a, c, g, or t
cgtgnatgagganccgcagtn

SEQ ID NO: 7 - DNA, 19 bp, Artificial Sequence
  Synthesized
  misc_feature (3)..(3): n is a, c, g, or t
  misc_feature (8)..(8): n is a, c, g, or t
  misc_feature (12)..(12): n is a, c, g, or t
  misc_feature (18)..(18): n is a, c, g, or t
gtnccttngcantctcant

SEQ ID NO: 8 - DNA, 20 bp, Artificial Sequence
  Synthesized
  misc_feature (3)..(3): n is a, c, g, or t
  misc_feature (9)..(9): n is a, c, g, or t
  misc_feature (14)..(14): n is a, c, g, or t
  misc_feature (19)..(19): n is a, c, g, or t
tgnatgagnagccncagtnt

SEQ ID NO: 9 - DNA, 19 bp, Artificial Sequence
  Synthesized
gtgccttggcagtctcagt

SEQ ID NO: 10 - DNA, 23 bp, Artificial Sequence
  Synthesized
acactgcgggctcctcatccacg

SEQ ID NO: 11 - DNA, 19 bp, Artificial Sequence
  Synthesized
gtgccttggcagtctcagu

SEQ ID NO: 12 - DNA, 22 bp, Artificial Sequence
  Synthesized
cgtggatgaggagccgcagtgu

SEQ ID NO: 13 - DNA, 19 bp, Artificial Sequence
  Synthesized
gtgccttggcagtcucagt

SEQ ID NO: 14 - DNA, 22 bp, Artificial Sequence
  Synthesized
cgtggatgaggagccgcagugt

SEQ ID NO: 15 - DNA, 18 bp, Artificial Sequence
  Synthesized
gtgccutggcaguctcag

SEQ ID NO: 16 - DNA, 21 bp, Artificial Sequence
  Synthesized
cgtggaugaggagcugcagtg